(19) United States
(12) Patent Application Publication
(10) Pub. No.: US 2014/0011769 A1
(43) Pub. Date: Jan. 9, 2014

(54) Methods of Treating Retroviral Infections and Related Dosage Regimes

(71) Applicant: Chimerix, Inc., Durham, NC (US)

(72) Inventor: Ernest Randall Lanier, Chapel Hill, NC (US)

(21) Appl. No.: 13/934,771

(22) Filed: Jul. 3, 2013

Related U.S. Application Data
(60) Provisional application No. 61/667,650, filed on Jul. 3, 2012.

(51) Int. Cl. A61K 31/675

(52) U.S. Cl. CPC A61K 31/675
USPC 514/81; 544/244

(57) ABSTRACT

The present invention relates to compounds and methods for treating retroviral infections, HIV, Hepatitis B, and/or HTLV viral infections. Some compounds of the invention are described by formula I:

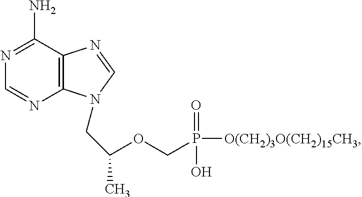

or a pharmaceutically acceptable salt, stereoisomer, a diastereomer, an enantiomer or racemate thereof.

### RELATED APPLICATIONS

This application claims priority to and the benefit of U.S. Provisional Application No. 61/667,650, filed Jul. 3, 2012, which is incorporated by reference herein in its entirety.

### FIELD OF THE INVENTION

Embodiments disclosed herein are directed to methods of treating retroviral infections with a phosphonate ester of tenofovir.

### BACKGROUND OF THE INVENTION

Tenofovir (TFV) disoproxil fumarate (TDF) is a widely used nucleoside/nucleotide reverse transcriptase inhibitors (NRTIs) approved for treatment of HIV. Upon administration, TDF is rapidly converted by plasma esterases to the tenofovir (TFV) dianion. Although TFV dianion is not readily taken up by target HIV infected cells, it is a substrate for organic anion transporters expressed at high levels on renal proximal tubule epithelial cells (RPTECs). TFV dianion uptake by RPTECs mediated by the organic anion transporters allows high intracellular effective concentration (˜50% (EC₅₀s)), which is associated with renal toxicity at a low frequency. Miller et al., J. Infect. Dis., 189:837-846 (2004), and Szczech et al., Top. HIV Med., 16:122-126 (2008).

One TFV derivative with lower renal toxicity is hexadecyloxypropyl tenofovir or HDP-TFV (3-(hexadecyloxy)propyl hydrogen ((R)-1-(6-amino-9H-purin 9-yl)propan-2-yloxy)methylphosphonate), which is a lipid conjugate of tenofovir (TFV)). HDP-TFV has the following formula:

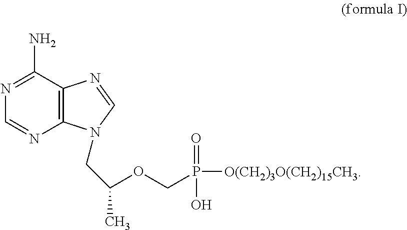

The cellular uptake of HDP-TFV is higher than TFV because the lipid allows uptake of the molecule to be achieved via a natural lipid uptake pathway, such as the lysophosphatidylcholine uptake pathway. Despite the increased cellular uptake, HDP-TFV does not adverse effects, such as renal toxicity, of TDF. Therefore, HDP-TFV provides an alternative to treating infections by HIV and other retroviruses with TDF. The present invention relates to the use of HDP-TFV for treating diseases caused by retroviruses, such as acquired immune deficiency syndrome (AIDS) caused by human immunodeficiency virus (HIV) and adult T-cell leukemia (ATL) caused by human T-cell lymphotropic virus-I (HTLV-I). The present invention also relates to the use of HDP-TFV for inhibiting replication of human T-cell lymphotropic virus-I (HTLV-I) in animal cells.

### SUMMARY OF THE INVENTION

In one embodiment, the present invention relates to a pharmaceutical composition for treating a viral infection or viral disease, comprising a compound having the formula:

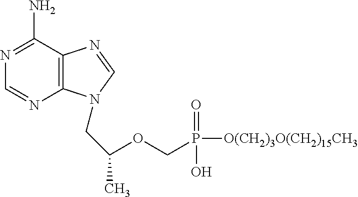

or a pharmaceutically acceptable salt thereof, wherein the viral infection or viral disease is treated in about three weeks after administration. In an embodiment, the compound decreases viral replication. In another embodiment, the viral infection is human T cell leukemia virus-1 (HTLV-I) infection.

In an embodiment, the present invention relates to a method for treating a viral infection or viral disease in a subject, the method comprising administering to the subject a composition comprising a compound having a formula:

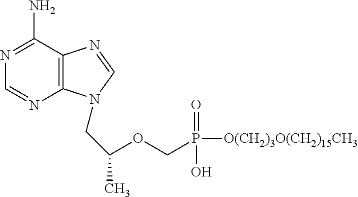

or a pharmaceutically acceptable salt thereof, wherein the compound is effective in treating the viral infection or viral disease in about three weeks after administration. In an embodiment, the method results in decreasing viral replication. In an embodiment, the virus is a retrovirus. In an embodiment, the viral infection or viral disease is an infection or disease of a human retrovirus selected from the group consisting of HIV-1, HIV-2, HTLV-I and HTLV-II. In an embodiment, the subject is a human being. In an embodiment, the administration is before acute viral infection. In an embodiment, the composition is administered before seroconversion. In an embodiment, the composition is administered after serocoversion.

The present invention relates to a method for inhibiting replication of reverse transcriptase dependent virus in animal cells, comprising administering to said cells a composition comprising a compound of having the formula:

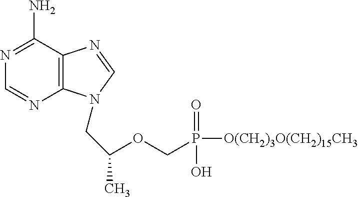

or a pharmaceutically acceptable salt thereof. In an embodiment, the compound is administered to cells in vivo. In another embodiment, the animal cells are mammalian cells. In an embodiment, the virus is a retrovirus. In an embodiment, the virus is a human retrovirus selected from the group consisting of HIV-1, HIV-2, HTLV-I and HTLV-II and said cells are human cells. In an embodiment, the composition is administered to a human being before acute viral infection. In an embodiment, the composition is administered to a human being before seroconversion. In an embodiment, the composition is administered to a human being after serocoversion.

The methods of the present invention provide higher concentrations of active antiviral (i.e., tenofovir diphosphate) in vivo using lower dosages of the compound of the invention relative to tenofovir administration.

### BRIEF DESCRIPTION OF THE DRAWINGS

FIGS. 1A-B are images of polymerase chain reaction (PCR) amplified HTLV-1 and human GAPDH DNA sequences from HTLV-1 infected PMBC cells treated for 2 (1A) and 4 (1B) weeks with AZT, tenofovir, and HDP-TFV.

FIGS. 2A-C show line graphs of data from an HTLV p19 Antigen ELISA after exposing cells to AZT (2A), tenofovir (2B), and HDP-TFV (2C), at concentrations between 0.1-25 μM.

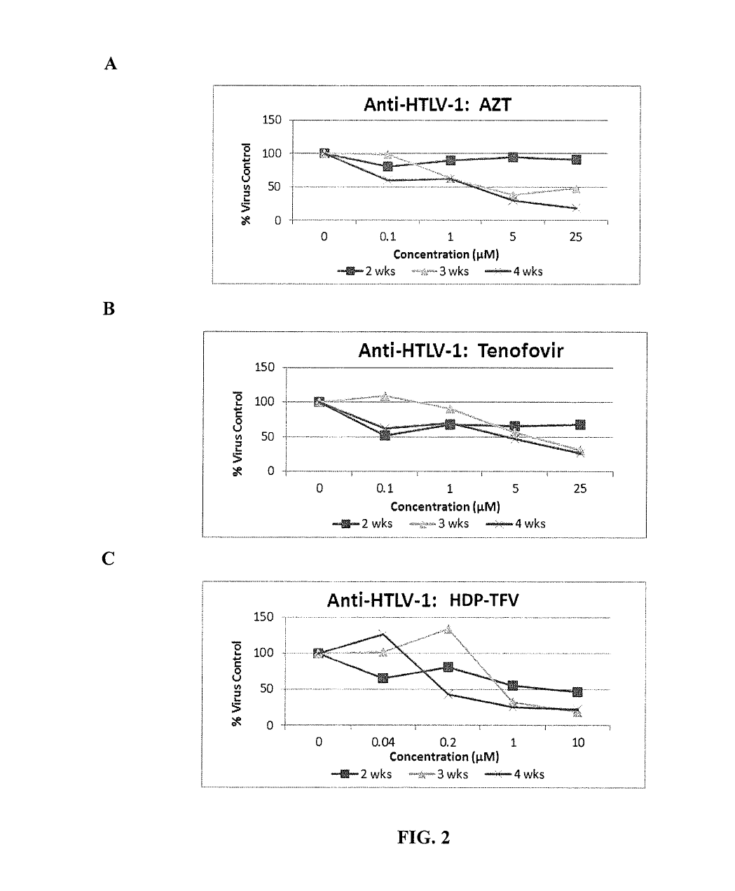

FIGS. 3A-B are images of polymerase chain reaction (PCR) amplified HTLV-1 and human GAPDH DNA sequences from HTLV-1 infected PMBC cells treated for 2 (3A) and 4 (3B) weeks with AZT, cifofovir, and HDP-CDV.

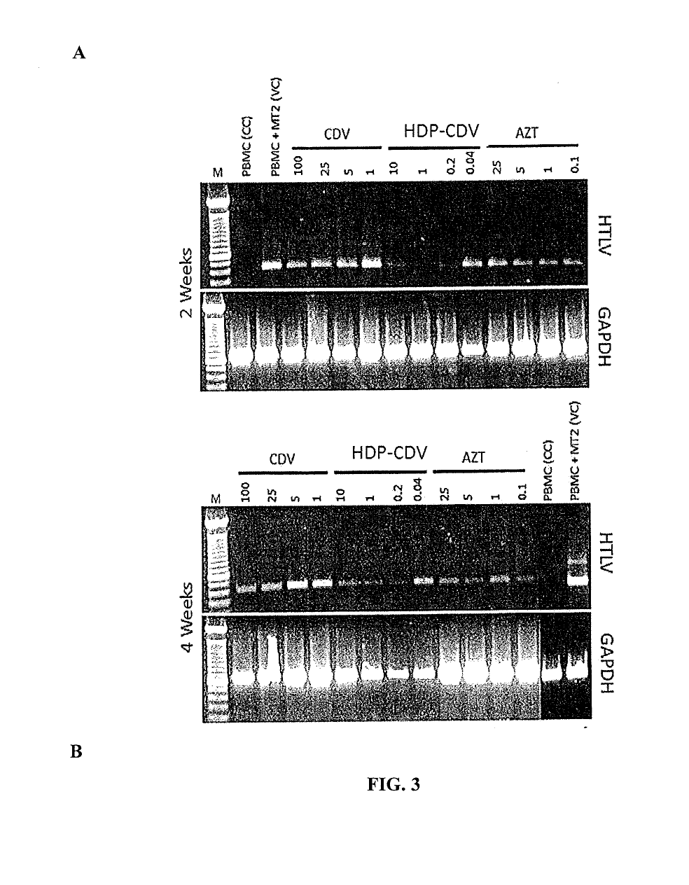

FIGS. 4A-C show line graphs of data from an HTLV p19 Antigen ELISA after exposing cells to AZT (4A), tenofovir (4B), and HDP-CDV (4C), at concentrations between 0.1-25 μM.

### DETAILED DESCRIPTION OF THE INVENTION

This invention is directed to the treatment of humans infected with HTLV-I or HTLV-II, including HTLV-I-associated leukemias and lymphomas, non-A, non-B hepatitis virus, hepatitis B virus, and Epstein-Barr virus (EBV), as well as to the treatment of animals infected with equine infectious anaemia or other lentiviruses.

The embodiments provide treating humans identified as having HTLV-I or HTLV-II infection, including HTLV-I-associated leukemias or lymphomas, non-A, non-B hepatitis, hepatitis B, or EBV infections, with a compound of the formula I, and/or a composition comprising a compound of the formula I:

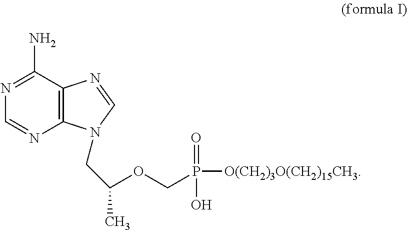

The embodiments of the current invention provide pharmaceutical compositions for treating a viral infection or viral disease, comprising a compound having the formula:

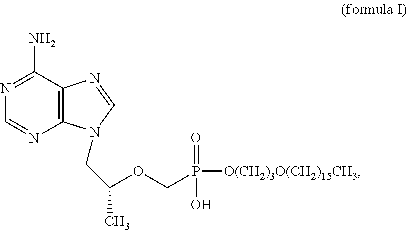

or a pharmaceutically acceptable salt thereof, wherein the viral infection or viral disease is treated in about three weeks after administration.

In one embodiment, the present invention relates to a compound having the formula:

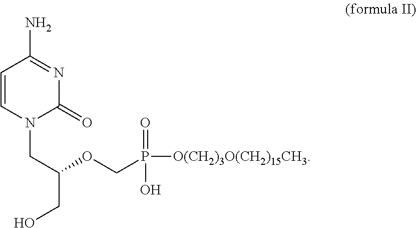

The compound of formula II is hexadecyloxypropyl cidofovir or HDP-CDV, which is a lipid conjugate of cidofovir. See e.g., US Patent Publication No. 2007/0003516; the contents of which are incorporated by reference herein.

### General Definitions

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this invention belongs.

The term “consists essentially of” (and grammatical variants), as applied to the compositions of this invention, means the composition can contain additional components as long as the additional components do not materially alter the composition. The term “materially altered,” as applied to a composition, refers to an increase or decrease in the therapeutic effectiveness of the composition of at least about 20% or more as compared to the effectiveness of a composition consisting of the recited components.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

All patents, patent applications and publications referred to herein are incorporated by reference in their entirety. In case of a conflict in terminology, the present specification is controlling.

As used herein, “alkali metals” are chemical elements from Group 1 of the periodic table of elements, for example: lithium (Li), sodium (Na), and potassium (K).

Subjects to be treated by the methods of the present invention are, in general, mammalian and primate subjects (e.g., human, monkey, ape, chimpanzee). Subjects may be male or female and may be of any age, including prenatal (i.e., in utero), neonatal, infant, juvenile, adolescent, adult, and geriatric subjects. Thus, in some cases the subjects may be pregnant female subjects.

As used herein, “Human immunodeficiency virus” (or “HIV”) as used herein is intended to include all subtypes thereof, including HIV subtypes A, B, C, D, E, F, G, and O, and HIV-2.

As used herein, “Hepatitis B virus” (or “HBV”) as used herein is intended to include all subtypes (adw, adr, ayw, and ayr) and or genotypes (A, B, C, D, E, F, G, and H) thereof.

As used herein, “human T-lymphotropic virus” (or “HTLV”) as used herein is intended to include all subtypes and or genotypes thereof. For example, HTLV Type I and HTLV Type II are included herein.

As used herein, “a therapeutically effective amount” refers to an amount that will provide some alleviation, mitigation, and/or decrease in at least one clinical symptom in the subject. Those skilled in the art will appreciate that the therapeutic effects need not be complete or curative, as long as some benefit is provided to the subject.

As used herein, “specificity” or “specifically against” refers to a compound that may selectively inhibit the metabolic activity and/or DNA replication of a certain type of viral infected cells. The specificity may be tested by using any methods known to one skilled in the art, for example, testing IC₉₀ and/or IC₅₀. In some embodiments, the compounds described herein may have IC₉₀ and/or IC₅₀ against viral infected cells to be at least about three fold lower than the IC₉₀ and/or IC₅₀ against normal (uninfected) cells. In some embodiments, the compounds described herein may have IC₉₀ and/or IC₅₀ against viral infected cells to be about three fold to ten-fold lower than the IC₉₀ and/or IC₅₀ against normal (uninfected) cells. In some embodiments, the compounds described herein may have IC₉₀ and/or IC₅₀ against viral infected cells to be at least ten fold lower than the IC₉₀ and/or IC₅₀ against normal (uninfected) cells. In some embodiments, the compounds described herein may have specific cytotoxicity against viral infected and/or transformed cells. The cytotoxicity may be measured by any methods known to one skilled in the art.

Unless otherwise stated, structures depicted herein are meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

“Treating”, includes any effect, e.g., lessening, reducing, modulating, or eliminating, that results in the improvement of the condition, disease, disorder, etc. “Treating” or “treatment” of a disease state includes: (1) inhibiting the disease state, i.e., arresting the development of the disease state or its clinical symptoms; (2) relieving the disease state, i.e., causing temporary or permanent regression of the disease state or its clinical symptoms; or (3) reducing or lessening the symptoms of the disease state.

In some embodiments, treatment may be administered after one or more symptoms have developed. In other embodiments, treatment may be administered in the absence of symptoms. Treatment may also be continued after symptoms have resolved.

As used herein, the terms “prevention,” “prevent,” and “preventing” refer to causing the clinical symptoms of the disease state not to develop in a subject that may be exposed to or predisposed to the disease state, but does not yet experience or display symptoms of the disease state. In some embodiments, prevention may be administered in the absence of symptoms. For example, prevention may be administered to a susceptible individual prior to the onset of symptoms (e.g., in light of a history of symptoms and/or in light of genetic or other susceptibility factors). Prevention may also be continued after symptoms have resolved, for example to delay their recurrence.

Active compounds of the present invention may optionally be administered in combination (or in conjunction) with other active compounds and/or agents useful in the treatment of viral infections as described herein. The administration of two or more compounds “in combination” or “in conjunction” means that the two or more compounds are administered closely enough in time to have a combined effect, for example an additive and/or synergistic effect. The two or more compounds may be administered simultaneously (concurrently) or sequentially or it may be two or more events occurring within a short time period before or after each other. Simultaneous administration may be carried out by mixing the compounds prior to administration, or by administering the compounds at the same point in time but at different anatomic sites or using different routes of administration. In some embodiments, the other antiviral agent(s) may optionally be administered concurrently.

“Parenteral” as used herein refers to subcutaneous, intravenous, intra-arterial, intramuscular or intravitreal injection, or infusion techniques.

“Topically” as used herein encompasses administration rectally and by inhalation spray, as well as the more common routes of the skin and mucous membranes of the mouth and nose and in toothpaste.

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### Pharmaceutical Compositions and Salts

The present invention includes compounds of formula I and pharmaceutically acceptable salts for use in the treatment of infections or diseases associated with HTLV-I. The composition comprising the compound of formula I or a pharmaceutically acceptable salt thereof may decrease viral replication. The compound of formula I or a pharmaceutically acceptable salt thereof may treat infection and reduce replication of human T cell leukemia virus-I (HTLV-I).

One aspect of the invention provides a compound of formula I:

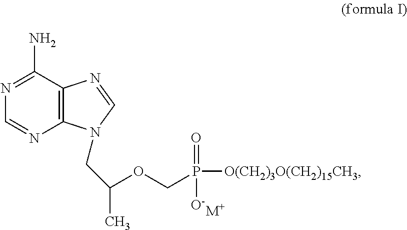

wherein M⁺ is potassium (K⁺), sodium (Na⁺), lithium (Li⁺), calcium (Ca²⁺), magnesium (Mg²⁺) or any pharmaceutically acceptable cation containing at least one nitrogen, or a stereoisomer, diastereomer, enantiomer or racemate thereof. Exemplary cations containing at least one nitrogen include, but are not limited to, various ammonium, mono, di, tri or tetra substituted amino cations. In one embodiment, the cations containing at least one nitrogen may be represented by the formula of [NR₁R₂R₃R₄]⁺ where R₁, R₂, R₃, and R₄ are independently hydrogen or an aliphatic moiety. In one embodiment, the aliphatic moiety is selected from C₁₋₅ alkyl (e.g., NH₄⁺, NH₃CH₃⁺, NH₃CH₂CH₃⁺, etc), C₂₋₅ alkenyl, or C₂₋₅ alkynyl, etc. In another embodiment, the compound of formula I is a salt selected from the group consisting of: methylamine, ethylamine, ethanolamine, tris(hydroxymethyl)aminomethane, ethylenediamine, dimethylamine, diethylamine, diisopropylamine, dibutylamine, di-sec-butylamine, dicyclohexylamine, diethanolamine, meglumine, pyrrolidine, piperidine, piperazine, benzathine, trimethylamine, triethylamine, triethanolamine, 1-(2-hydroxyethyl)-pyrrolidine, choline, tetra-methylammonium, and tetraethylammonium. For compounds of formula I, when M⁺ is Ca²⁺ or Mg²⁺, two equivalents of the anion are present to meet the requirement for cation-anion balance.

In one embodiment, the compound is:

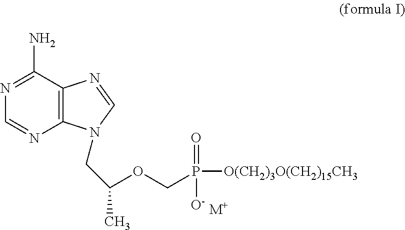

where M⁺ may be, e.g., K⁺.

The salt may be in various forms, all of which are included within the scope of the invention. These forms include anhydrous form or solvates. In one embodiment, M⁺ is K⁺. In other embodiments, the salt may be crystalline.

In one embodiment, the present invention is a pharmaceutical composition comprising a compound described herein. In another embodiment, the pharmaceutical composition further comprises a pharmaceutically acceptable carrier. The term “pharmaceutically acceptable carrier” as used herein refers to any substance, not itself a therapeutic agent, used as a vehicle for delivery of a therapeutic agent to a subject. Examples of pharmaceutically acceptable carriers and methods of manufacture for various compositions include, but are not limited to, those described in Remington's Pharmaceutical Sciences, 18th Ed., Mack Publishing Co. (1990) (See also US Patent Application US 2007/0072831).

The compounds of the invention may be formulated with conventional carriers, diluents and excipients, which will be selected in accord with ordinary practice. Tablets will contain excipients, glidants, fillers, binders, diluents and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Another aspect of the invention provides a pharmaceutical composition, wherein said composition is in the dosage form of a tablet or a capsule, an intravenous formulation, a solution, or a suspension comprising a compound described herein.

### Preparation of Compositions

The present invention includes use of compounds of formula I and pharmaceutically acceptable salts in the preparation of pharmaceutical formulations for the treatment of HTLV-I infection. The above-mentioned pharmaceutically acceptable salts may be prepared in a conventional manner, e.g., treatment of the compound with an appropriate base.

In general, the compounds of this invention may be prepared by standard techniques known in the art and by known processes analogous thereto. For example, HDP-TFV may be prepared in accordance with known procedures, or variations thereof that will be apparent to those skilled in the art. See, e.g., Painter et al., Antimicrobial Agents and Chemotherapy 51, 3505-3509 (2007) and US Patent Application Publication No. 2007/0003516 to Almond et al; their contents are incorporated by reference herein.

Specifically, general methods for preparing compounds of the present invention are set forth below. In the following description, all variables are, unless otherwise noted, as defined in the formulas described herein. The following non-limiting descriptions illustrate the general methodologies that may be used to obtain the compounds described herein.

In one embodiment, the pharmaceutically acceptable salts described herein may be prepared by dissolving the compound of formula I in an appropriate solvent,

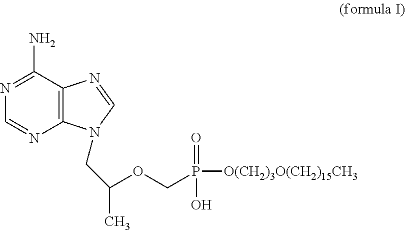

adding a suitable base to the mixture of the solvent and the compound of formula I, and removing the solvent to provide the compound of formula I.

A further aspect of the invention provides processes of preparing a pharmaceutically acceptable salt described herein. The processes comprise dissolving the compound of formula I in a solvent,

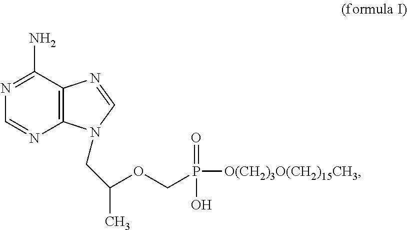

to form a solution, adding a base to the solution to form the salt, and removing the solvent.

The solvent used in the preparation may be any suitable solvent known to one skilled in the art or a combination of solvents that provides satisfactory yield of the product. In one embodiment, the solvent is a mixture of at least two solvents. Exemplary combination of solvents includes, but is not limited to, dichloromethane and methanol, dichloromethane and ethanol. In one embodiment, the molar ratio of the dichloromethane and methanol is in a range of about 1:1 to 9:1. In one embodiment, the molar ratio of the dichloromethane and methanol is in a range of about 7:3 to 9:1. In a further embodiment, the molar ratio of the dichloromethane and methanol is about 9:1.

The base used in the preparation may be any suitable base known to one skilled in the art or a combination of bases that provides satisfactory yield of the product. In some embodiments, the base is an alkali metal alcoholate base. Exemplary bases include, but are not limited to, potassium methoxide, sodium methoxide, lithium tert-butoxide, ammonium hydroxide, sodium hydroxide, potassium hydroxide, and lithium hydroxide.

The process described herein may further include the step of recrystallization to remove impurity, side products, and unreacted starting material. The recrystallization step comprises the step of dissolving the product in a suitable solvent at an appropriate temperature, cooling to an appropriate temperature for a sufficient period of time to precipitate the compound, and filtering to provide the compound of formula I. In some embodiments, the temperature for the step of dissolving is in a range of about 50° C. to 80° C.

### Treating Infections

Embodiments of the current invention include methods of treating or preventing a viral disease. The methods comprises administering to a subject an effective amount of a compound described herein. In one embodiment, the virus is a retrovirus, e.g., human immunodeficiency virus (HIV) or xenotropic murine leukemia virus-related virus (XMRV). In another embodiment, the virus is Hepatitis B virus (HBV). In another embodiment, the virus is human T-lymphotropic virus (HTLV), e.g., HTLV Type I. In one embodiment, the virus is HTLV Type II.

A further aspect of the invention relates to methods of treating a subject infected with at least one retrovirus and the subject has not been administered an antiviral active agent for the retrovirus. The invention provides methods of treating a subject infected with HBV who has not been administered an antiviral active agent for HBV. The methods comprise administering a compound described herein to the infected subject in an amount effective to treat the viral infection and inhibit the development of resistance to an antiviral compound.

Another aspect of the invention includes methods of treating a subject infected with at least one retrovirus and, in response to prior administration of an antiviral compound, has developed resistance or a toxic response to at least one other antiviral compound. In another aspect, the embodiments of the invention provide methods of treating a subject infected with HTLV-I or HTLV-II and, in response to prior administration of an antiviral compound, the subject has developed resistance or a toxic response to at least one other anti-viral compound. The methods comprise administering to the infected subject a compound described herein in an amount effective to treat the viral infection and inhibit the further development of resistance to the antiviral compound in the infected subject.

In an embodiment, the present invention relates to a method for treating a viral infection or viral disease in a subject, the method comprising administering to the subject a composition comprising a compound having a formula:

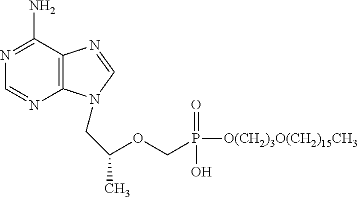

or a pharmaceutically acceptable salt thereof, wherein the compound is effective in treating the viral infection or viral disease in about three weeks after administration. In an embodiment, the method results in decreasing viral replication. In an embodiment, the virus is a retrovirus. In an embodiment, the viral infection or viral disease is an infection or disease of a human retrovirus selected from the group consisting of HIV-1, HIV-2, HTLV-I and HTLV-II. In an embodiment, the subject is a human being. In an embodiment, the administration is before acute viral infection. In an embodiment, the composition is administered before seroconversion. In an embodiment, the composition is administered after serocoversion.

The present invention relates to a method for inhibiting replication of reverse transcriptase dependent virus in animal cells, comprising administering to said cells a composition comprising the compound of having a formula:

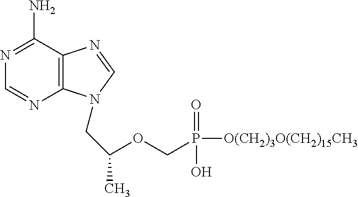

or pharmaceutically acceptable salt thereof. In an embodiment, the compound is administered to cells in vivo. In another embodiment, the animal cells are mammalian cells. In an embodiment, the virus is a retrovirus. In an embodiment, the virus is a human retrovirus selected from the group consisting of HIV-1, HIV-2, HTLV-I and HTLV-II and said cells are human cells. In an embodiment, the composition is administered to a human being before acute viral infection. In an embodiment, the composition is administered to a human being before seroconversion. In an embodiment, the composition is administered to a human being after serocoversion.

The methods of the present invention provide higher concentrations of active antiviral (i.e., tenofovir diphosphate) in vivo using lower dosages of the compound of the invention relative to tenofovir.

The compound and/or composition comprising the compound of formula I is useful in treating animals identified as having equine infectious anaemia or other lentivirus infections.

A further aspect of the invention provides methods of inhibiting sexual transmission of HIV. The methods comprise topically applying to the skin or epithelial tissue of a human a therapeutically effective amount of a composition comprising the compound described herein. The methods further comprise concurrently administering the subject one or more additional antiviral active agents with the compound described herein.

In accordance with one aspect of the invention, provided are methods for treating disorders caused by viral infections. In some aspects of the invention, the virus is a retrovirus. In one embodiment, the virus is a gamma retrovirus. As used herein, “retrovirus” is an RNA virus that is replicated in a host cell via the enzyme reverse transcriptase to produce DNA from its RNA genome. The DNA is then incorporated into the host's genome by an integrase enzyme. The virus thereafter replicates as part of the host cell's DNA. Retroviruses are enveloped viruses that belong to the viral family Retroviridae. Exemplary retroviruses include, but are not limited to, human immunodeficiency virus (HIV) and xenotropic murine leukemia virus-related virus (XMRV). In addition, there is evidence to indicate that XMRV may be related to chronic fatigue syndrome (CFS). (See, e.g., Lombardi, et al., Science, vol. 326, P 585-589 (October 2009).) Compounds of the invention are useful in treating HIV, XMRV, or CFS.

In another embodiment, the invention provides a method of treating or preventing an XMRV infection comprising administering to a subject an effective amount of a compound of the invention. In another embodiment, the invention provides a method of treating or preventing chronic fatigue syndrome comprising administering to a subject an effective amount of a compound of the invention. In another embodiment, the invention provides a method of treating or preventing prostate cancer comprising administering to a subject an effective amount of a compound of the invention.

In another embodiment, the invention provides a method of treating or preventing a hepatitis B infection comprising administering to a subject an effective amount of a compound of the invention.

In one embodiment, the subject is human. In one embodiment, the subject is an immunocompromised and/or an immunosuppressed subject. In some embodiments, the toxic side effects in the immunodeficient subject are decreased when using the methods of the present invention, compared to the toxic side effects of using tenofovir or other antiviral agents.

As used herein, immunodeficiency (or immune deficiency) is a state in which the immune system's ability to fight infectious disease is compromised or entirely absent. An immunocompromised subject is a subject that has an immunodeficiency of any kind or of any level. Exemplary immunocompromised subject includes, but are not limited to, a subject with primary immunodeficiency (a subject that is born with defects in immune system) and a subject with secondary (acquired) immunodeficiency In addition, other common causes for secondary immunodeficiency include, but are not limited to, malnutrition, aging and particular medications (e.g. immunosuppressive therapy, such as chemotherapy, disease-modifying antirheumatic drugs, immunosuppressive drugs after organ transplants, glucocorticoids). Other exemplary diseases that directly or indirectly impair the immune system include, but are not limited to, various types of cancer, (e.g. bone marrow and blood cells (leukemia, lymphoma, multiple myeloma)), acquired immunodeficiency syndrome (AIDS) caused by human immunodeficiency virus (HIV), chronic infections and autoimmune diseases (e.g. Acute disseminated encephalomyelitis (ADEM), Addison's disease, Alopecia areata, Ankylosing spondylitis, Antiphospholipid antibody syndrome (APS), Autoimmune hemolytic anemia, Autoimmune hepatitis, Autoimmune inner ear disease, Bullous pemphigoid, Coeliac disease, Chagas disease, Chronic obstructive pulmonary disease, Crohns Disease, Dermatomyositis, Diabetes mellitus type 1, Endometriosis, Goodpasture's syndrome, Graves' disease, Guillain-Barré syndrome (GBS), Hashimoto's disease, Hidradenitis suppurativa, Kawasaki disease, IgA nephropathy, Idiopathic thrombocytopenic purpura, Interstitial cystitis, Lupus erythematosus, Mixed Connective Tissue Disease, Morphea, Multiple sclerosis (MS), Myasthenia gravis, Narcolepsy, Neuromyotonia, Pemphigus vulgaris, Pernicious anaemia, Psoriasis, Psoriatic Arthritis, Polymyositis, Primary biliary cirrhosis, Rheumatoid arthritis, Schizophrenia, Scleroderma, Sjögren's syndrome, Stiff person syndrome, Temporal arteritis (also known as “giant cell arteritis”), Ulcerative Colitis, Vasculitis, Vitiligo, Wegener's granulomatosis.)

The antiviral activity for HDP-TFV has been described in e.g., U.S. Pat. Nos. 6,716,825, 7,034,014, 7,094,772, 7,098,197, 7,452,898, and in PCT publication No. WO 2008/133966, which are incorporated by reference in their entireties.

It has also been found that compounds described herein may associate with or bind to viral particles. Since viral particles migrate or permeate into cellular or tissue compartments that are not generally accessible to active therapeutic agents (thus creating a substantially untreated “reservoir” of infection when subjects are systemically administered such agents), this finding makes possible (a) the treatment of infection in such privileged compartments, and (b) the use of active agents in prophylactic or microbicidal treatments (where association or binding of the active agent to virus before infection occurs is of therapeutic benefit).

In general, a privileged compartment is a cellular or tissue compartment to which said virus permeates in vivo, to which said active agent does not efficiently permeate in vivo in the absence of said virus, and to which said active agent is carried in vivo by said virus when said active agent binds to said virus. For example, when the privileged compartment is a tissue compartment, it may be brain (central nervous system), lymphoid, or testes. Examples of cellular privileged compartments include but are not limited to dendritic cells, microglia, monocyte/macrophages, and combinations thereof. Compositions and methods of treating privileged compartment infections may be prepared and carried out as described above. Prophylactic compositions, devices and methods are discussed in further detail below.

The treatment for privileged compartment infections using HDP-TFV has been described in PCT Publication Nos. WO 2009/094191 and WO 2009/094190, which are incorporated by reference in their entireties.

### Additional Antiviral Agents for Combination Therapy

In combination with compounds of the invention, additional antiviral active agents that may be used in carrying out the present invention include HIV-protease inhibitors, nucleoside reverse transcriptase inhibitors (this term herein including nucleotide reverse transcriptase inhibitors), non-nucleoside reverse transcriptase inhibitors, integrase inhibitors, entry inhibitors, fusion inhibitors, maturation inhibitors, and combinations thereof. Numerous examples are known and described in, for example, US Patent Application Publication No. 2006/0234982 to Dahl et al. at Table A therein, and in Table 1 as set forth below.

Additional antiviral active agents that may be used in carrying out the present invention include ribavirin, interferon (e.g., interferon alpha, pegylated interferon), lamivudine, entecavir, telbivudine, emtricitabine, clevudine, BAM-205 (NOV-205), LB80380, MIV-210 (lagociclovir valactate), simvastatin, Bay 41-4109 and combinations thereof.

Additional examples include, but are not limited to, the integrase inhibitor Isentress or raltegravir (MK-0518: Merck), the CCR5 inhibitor Maraviroc or selzentry (and K-427857, Pfizer) and others of these classes.

Additional examples are provided in U.S. Pat. No. 7,094,413 to Buelow et al.; U.S. Pat. No. 7,250,421 to Nair et al., US Patent Application Publication No. 2007/0265227 to Heneine et al. and US Patent Application Publication No. 2007/0072831 to Cai et al.

The non-nucleoside reverse transcriptase inhibitor (“NNRTI”) 6-chloro-4-cyclopropylethynyl-4-trifluoromethyl-1,4-dihydro-2H3,1-benzoxazin-2-one, and pharmaceutically acceptable salts thereof, are described in, for example, U.S. Pat. No. 5,519,021. Examples of the present invention include efavirenz.

The nucleoside reverse transcriptase inhibitor (“NRTI”) 2-hydroxymethyl-5-(5-fluorocytosin-1-yl)-1,3-oxathiolane (“FTC”) and pharmaceutically acceptable salts thereof, are described in, for example, U.S. Pat. No. 6,642,245 to Liotta et al. Examples of the present invention include emtricitabine.

Integrase inhibitors include, but are not limited to, those described in US Patent Application Publication No. 2007/0072831, WO 02/30426, WO 02/30930, WO 02/30931, WO 02/055079, WO 02/36734, U.S. Pat. No. 6,395,743; U.S. Pat. No. 6,245,806; U.S. Pat. No. 6,271,402; WO 00/039086; WO 00/075122; WO 99/62513; WO 99/62520; WO 01/00578; Jing, et al., Biochemistry, 41, 5397-5403, (2002); Pais, et al., J. Med. Chem., 45, 3184-94 (2002); Goldgur, et al., Proc. Natl. Acad. Sci. U.S.A., 96, 13040-13043 (1999); Espeseth, et al., Proc. Natl. Acad. Sci. U.S.A., 97,11244-11249, (2000); WO 2005/016927, WO 2004/096807, WO 2004/035577, WO 2004/035576 and US 2003/0055071.

TABLE 1
Additional Antiviral Agents

5,6 dihydro-5-azacytidine
5-aza 2′deoxycytidine
5-azacytidine
5-yl-carbocyclic 2′-deoxyguanosine (BMS200,475)
9-(arabinofuranosyl)guanine; 9-(2′-deoxyribofuranosyl)guanine
9-(2′-deoxy-2′-fluororibofuranosyl)-2,6-diaminopurine
9-(2′-deoxy-2′-fluororibofuranosyl)guanine
9-(2′-deoxyribofuranosyl)-2,6-diaminopurine
9-(arabinofuranosyl)-2,6-diaminopurine
Abacavir, Ziagen ®
Acyclovir, ACV; 9-(2-hydroxyethoxylmethyl)guanine
Adefovir dipivoxil, Hepsera ®
Amdoxivir, DAPD
Amprenavir, Agenerase ®
araA; 9-β-D-arabinofuranosyladenine (Vidarabine)
Atazanivir sulfate (Reyataz ®)
AZT; 3′-azido-2′,3′-dideoxythymdine, Zidovudine, (Retrovir ®)
BHCG;(+−)-(1a,2b,3a)-9-[2,3-bis(hydroxymethyl)cyclobutyl]guanine
BMS200,475; 5-yl-carbocyclic 2′-deoxyguanosine
Buciclovir; (R) 9-(3,4-dihydroxybutyl)guanine
BvaraU; 1-β-D-arabinofuranosyl-E-5-(2-bromovinyl)uracil (Sorivudine)
Calanolide A
Capravirine
CDG; carbocyclic 2′-deoxyguanosine
Cidofovir, HPMPC; (S)-9-(3-hydroxy-2-
phosphonylmethoxypropyl)cytosine
Clevudine, L-FMAU; 2′-Fluoro-5-methyl-β-L-arabino-furanosyluracil
Combivir ® (lamivudine/zidovudine)
Cytallene; [1-(4′-hydroxy-1′,2′-butadienyl)cytosine]
DAPD; (−)-β-D-2,6-diaminopurine dioxolane
ddA; 2′,3′-dideoxyadenosine
ddAPR; 2,6-diaminopurine-2′,3′-dideoxyriboside
ddC; 2′,3′-dideoxycytidine (Zalcitabine)
ddI; 2′,3′-dideoxyinosine, didanosine, (Videx ®, Videx ® EC)
Delavirdine, Rescriptor ®
Didanosine, ddI, Videx ®; 2′,3′-dideoxyinosine
DXG; dioxolane guanosine
E-5-(2-bromovinyl)-2′-deoxyuridine
Efavirenz, Sustiva ®
Enfuvirtide, Fuzeon ®
F-ara-A; fluoroarabinosyladenosine (Fludarabine)
FDOC; (−)-β-D-5-fluoro-1-[2-(hydroxymethyl)-1,3-dioxolane]cytosine
FEAU; 2′-deoxy-2′-fluoro-1-β-D-arabinofuranosyl-5-ethyluracil
FIAC; 1-(2-deoxy-2-fluoro-β-D-ababinofuranosyl)-5-iodocytosine
FIAU; 1-(2-deoxy-2-fluoro-β-D-ababinofuranosyl(-5-iodouridine
FLG; 2′,3′-dideoxy-3′-fluoroguanosine
FLT; 3′-deoxy-3′-fluorothymidine
Fludarabine; F-ara-A; fluoroarabinosyladenosine
FMAU; 2'-Fluoro-5-methyl-β-L-arabino-furanosyluracil
FMdC
Foscarnet; phosphonoformic acid, PFA
FPMPA; 9-(3-fluoro-2-phosphonylmethoxypropyl)adenine
Gancyclovir, GCV; 9-(1,3-dihydroxy-2-propoxymethyl)guanine
GS-7340; 9-[R-2-[[(S)-[[(S)-1-(isopropoxycarbonyl)ethyl]amino]-
phenoxyphosphinyl methoxy]propyl]adenine
HPMPA; (S)-9-(3-hydroxy-2-phosphonylmethoxypropyl)adenine
HPMPC; (S)-9-(3-hydroxy-2-phosphonylmethoxypropyl)cytosine
(Cidofovir)
Hydroxyurea, Droxia ®
Indinavir, Crixivan ®
Kaletra ® (lopinavir/ritonavir)
Lamivudine, 3TC, Epivir ™; (2R,5S,cis)-4-amino-1-(2-hydroxymethyl-
1,3-oxathiolan-5-yl)-(1H)-pyrimidin-2-one
L-d4C; L-3′-deoxy-2′,3′-didehydrocytidine
L-ddC; L-2′,3′-dideoxycytidine
L-Fd4C; L-3′-deoxy-2′,3′-didehydro-5-fluorocytidine
L-FddC; L-2′,3′-dideoxy-5-fluorocytidine
Lopinavir
Nelfinavir, Viracept ®
Nevirapine, Viramune ®
Oxetanocin A; 9-(2-deoxy-2-hydroxymethyl-β-D-erythro-
oxetanosyl)adenine
OxetanocinG;9-(2-deoxy-2-hydroxymethyl-β-D-erythro-
oxetanosyl)guanine
Penciclovir
PMEDAP; 9-(2-phosphonylmethoxyethyl)-2,6-diaminopurine
PMPA, tenofovir; (R)-9-(2-phosphonylmethoxypropyl)adenine
PPA; phosphonoacetic acid
Ribavirin; 1-β-D-ribofuranosyl-1,2,4-triazole-3-carboxamide
Ritonavir, Norvir ®
Saquinavir, Invirase ®, Fortovase ®
Sorivudine, BvaraU; 1-β-D-arabinofuranosyl-E-5-(2-bromovinyl)uracil
Stavudine, d4T, Zerit ®; 2′,3′-didehydro-3′-deoxythymidine
Trifluorothymidine, TFT;
Trizivir ® (abacavir sulfate/lamivudine/zidovudine)
Vidarabine, araA; 9-β-D-arabinofuranosyladenine
Viread ®, tenofovir disoproxil fumarate (DF), Bis POC PMPA, TDF;
2,4,6,8-Tetraoxa-5-phosphanonanedioic acid, 5-[[(1R)-2-(6-amino-9H-
purin-9-yl)-1-methylethoxy]methyl]-, bis(1-methylethyl)ester,
5-oxide, (2E)-2-butenedioate (1:1)
Zalcitabine, Hivid ®, ddC; 2′,3′0dideoxycytidine
Zidovudine, AZT, Retrovir ®; 3′-azido-2′,3′-dideoxythymidine
Zonavir; 5-propynyl-1-arabinosyluracil
Rilpivirine (TMC278)

In another embodiment, the compositions of the present invention can include an active compound as described herein in combination with one or more (e.g., 1, 2, 3, or more) additional active agents described above. Specific examples of such combinations include, but are not limited to: a compound described herein in combination with:
(a) FTC/Efavirenz;
(b) 3TC/Efavirenz;
(c) AZT/3TC;
(d) FTC;
(e) 3TC;
(f) FTC/Isentress;
(g) 3TC/Isentress;
(h) PPL-100;
(i) FTC/TMC278;
(j) 3TC/TMC278;
(k) FTC/TMC125; or
(l) 3TC/TMC125.

### Delivery—Routes and Dosage Forms

The compound of formula I may be administered as a pure form or in the form of a pharmaceutically acceptable salt to the infected animal or human, e.g., an alkali metal salt such as sodium or potassium salts, an alkaline earth metal salt or an ammonium salt such as tetraalkylammonium salts (all of which are hereinafter referred to as a pharmaceutically acceptable base salt).

Another aspect of the invention provides a pharmaceutical composition comprising the compound described herein and at least one additional antiviral active agent and a pharmaceutically acceptable carrier.

Preferably the compound of the invention is administered orally, preferably at a dosage of from about 1 mg/kg to about 100 mg/kg, more preferably at a dosage of from about 1 mg/kg to about 20 mg/kg. For example, said compound is administered to said subject at a dosage of 1, 2, 3, 4, 5, 6, 7, 8, 9, 10, 11, 12, 13, 14, 15, 16, 17, 18, 19 or 20 mg/kg. In addition, said compound is administered to said subject in an amount of about 25, 50, 75, 100, 125, 150, 175, 200, 250, 300, 350, 400, 450, 500, 600, 700, 800, 900, 1000, 1100, 1200, 1300, 1400, 1500, 1600, 1700, 1800, 1900 or 2000 mg. The compounds of the invention can be administered, for example, as a single dose, daily, or weekly.

In another embodiment, the compounds describe herein can be administered, for example, as a single dose, weekly, or every other week, or every three weeks, or monthly. In another embodiment, the compounds describe herein can be administered in combination with an integrase inhibitor. In another embodiment, the compounds describe herein can be administered in combination with an integrase inhibitor for example, as single doses, weekly, or every other week, or every three weeks, or monthly.

In one embodiment, the compound which is orally administered is:

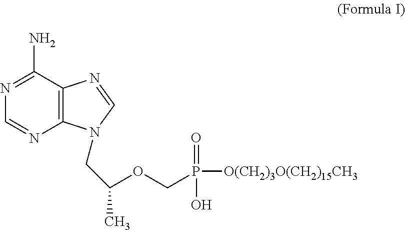

or a pharmaceutically acceptable salt thereof.

With respect to disorders associated with viral infections, the “effective amount” is determined with reference to the recommended dosages of the antiviral compound. The selected dosage will vary depending on the activity of the selected compound, the route of administration, the severity of the condition being treated, and the condition and prior medical history of the patient being treated. However, it is within the skill of the art to start doses of the compound(s) at levels lower than required to achieve the desired therapeutic effect and to gradually increase the dosage until the desired effect is achieved. If desired, the effective daily dose may be divided into multiple doses for purposes of administration, for example, two to four doses per day. It will be understood, however, that the specific dose level for any particular patient will depend on a variety of factors, including the body weight, general health, diet, time, and route of administration and combination with other drugs, and the severity of the disease being treated.

The compounds of the invention can be administered, for example, once per day for 1, 2, 3, 4, 5, 6, 7, 8, 9, or 10 days or more. For example, 25 mg of a compound of the invention can be administered daily. For example, 50 mg of a compound of the invention can be administered daily. For example, 100 mg of a compound of the invention can be administered daily. For example, 150 mg of a compound of the invention can be administered daily. For example, 200 mg of a compound of the invention can be administered daily. For example, 400 mg of a compound of the invention can be administered daily.

The compounds of the invention can be administered, for example, once per week for 1, 2, 3, 4, 5, 6, 7, 8, 9, or 10 weeks or more. For example, 25 mg of a compound of the invention can be administered weekly. For example, 50 mg of a compound of the invention can be administered weekly. For example, 100 mg of a compound of the invention can be administered weekly. For example, 150 mg of a compound of the invention can be administered weekly. For example, 200 mg of a compound of the invention can be administered weekly. For example, 250 mg of a compound of the invention can be administered weekly. For example, 300 mg of a compound of the invention can be administered weekly. For example, 350 mg of a compound of the invention can be administered weekly. For example, 400 mg of a compound of the invention can be administered weekly. For example, 450 mg of a compound of the invention can be administered weekly. For example, 500 mg of a compound of the invention can be administered weekly. For example, 750 mg of a compound of the invention can be administered weekly. For example, 1000 mg of a compound of the invention can be administered weekly. For example, 1250 mg of a compound of the invention can be administered weekly. For example, 1500 mg of a compound of the invention can be administered weekly. For example, 1750 mg of a compound of the invention can be administered weekly. For example, 2000 mg of a compound of the invention can be administered weekly.

Compounds of the invention (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient. The present invention further provides veterinary compositions comprising at least one active ingredient as above defined together with a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

The present invention can take the form of a topical compositions containing the active agents described herein for inhibiting or combating viral infection, e.g., for prophylactic use. Such compositions (with active agents other than those disclosed herein) are known and described in, for example, U.S. Pat. No. 6,545,007, the disclosure of which is incorporated herein by reference in its entirety.

Such compositions can take several forms. Thus, in one embodiment, the composition is in the form of a cream, lotion, gel, or foam that is applied to the affected skin or epithelial cavity, and preferably spread over the entire skin or epithelial surface which is at risk of contact with bodily fluids. Such formulations, which are suitable for vaginal or rectal administration, may be present as aqueous or oily suspensions, solutions or emulsions (liquid formulations) containing in addition to the active ingredient, such carriers as are known in the art to be appropriate. For “stand-alone” lubricants (i.e., lubricants that are not pre-packaged with condoms), gels and similar aqueous formulations are generally preferred, for various reasons (both scientific and economic) known to those skilled in the art. These formulations are useful to protect not only against sexual transmission of HIV, but also to prevent infection of a baby during passage through the birth canal. Thus the vaginal administration can take place prior to sexual intercourse, during sexual intercourse, and immediately prior to childbirth.

One method of applying an antiviral lubricant to the genitals, for the purposes disclosed herein, involves removing a small quantity (such as a teaspoon, or several milliliters) of a gel, cream, ointment, emulsion, or similar formulation from a plastic or metallic tube, jar, or similar container, or from a sealed plastic, metallic or other packet containing a single dose of such composition, and spreading the composition across the surface of the penis immediately before intercourse. Alternate methods of emplacement include: (1) spreading the composition upon accessible surfaces inside the vagina or rectum shortly before intercourse; and (2) emplacing a condom, diaphragm, or similar device, which has already been coated or otherwise contacted with an anti-viral lubricant, upon the penis or inside the vagina. In a preferred embodiment, any of these methods of spreading an anti-viral lubricant across the surfaces of the genitals causes the lubricant to coat and remain in contact with the genital and epithelial surfaces throughout intercourse.

In one embodiment the compositions are used in conjunction with condoms, to enhance the risk-reducing effectiveness of condoms and provide maximum protection for users. The composition can either be coated onto condoms during manufacture, and enclosed within conventional watertight plastic or foil packages that contain one condom per package, or it can be manually applied by a user to either the inside or the outside of a condom, immediately before use.

As used herein, “condom” refers to a barrier device which is used to provide a watertight physical barrier between male and female genitalia during sexual intercourse, and which is removed after intercourse. This term includes conventional condoms that cover the penis; it also includes so-called “female condoms” which are inserted into the vaginal cavity prior to intercourse. The term “condom” does not include diaphragms, cervical caps or other barrier devices that cover only a portion of the epithelial membranes inside the vaginal cavity. Preferably, condoms should be made of latex or a synthetic plastic material such as polyurethane, since these provide a high degree of protection against viruses.

In another embodiment the composition is in the form of an intra-vaginal pill, an intra-rectal pill, or a suppository. The suppository or pill should be inserted into the vaginal or rectal cavity in a manner that permits the suppository or pill, as it dissolves or erodes, to coat the vaginal or rectal walls with a prophylactic layer of the anti-HIV agent.

In still another embodiment the composition is topically applied by release from an intravaginal device. Devices such as vaginal rings, vaginal sponges, diaphragms, cervical caps, female condoms, and the like can be readily adapted to release the composition into the vaginal cavity after insertion.

Compositions used in the methods of this invention may also comprise additional active agents, such as another agent(s) to prevent HIV infection, and agents that protect individuals from conception and other sexually transmitted diseases. Thus, in another embodiment, the compositions used in this invention further comprise one or more additional anti-HIV agents, virucides effective against viral infections other than HIV, and/or spermicides.

In one particular embodiment, the composition contains nonoxynol, a widely-used spermicidal surfactant. The resulting composition could be regarded as a “bi-functional” composition, since it would have two active agents that provide two different desired functions, in a relatively inert carrier liquid; the nonoxynol would provide a spermicidal contraceptive agent, and the compound of the invention (i.e., HDP-TFV or a pharmaceutically acceptable salt thereof) would provide anti-viral properties. The nonoxynol is likely to cause some level of irritation, in at least some users; this is a well-known side effect of spermicidal surfactants such as nonoxynol and octoxynol, which attack and destroy the lipid bilayer membranes that surround sperm cells and other mammalian cells.

The compositions used in this invention may also contain a lubricant that facilitates application of the composition to the desired areas of skin and epithelial tissue, and reduces friction during sexual intercourse. In the case of a pill or suppository, the lubricant can be applied to the exterior of the dosage form to facilitate insertion.

In still another embodiment the invention provides a device for inhibiting the sexual transmission of HIV comprising (a) a barrier structure for insertion into the vaginal cavity, and (b) a composition comprising an active agent as described herein. As mentioned above, preferred devices which act as barrier structures, and which can be adapted to apply anti-HIV agent, include the vaginal sponge, diaphragm, cervical cap, or condom (male or female).

The methods, compositions and devices of this invention can be adapted generally to release active agent in a time sensitive manner that best corresponds to the timing of sexual activity. When topically applied as a lotion or gel, the compositions are preferably applied immediately prior to sexual activity. Other modes of application, such as devices and suppositories, can be designed to release active agent over a prolonged period of time, at a predetermined rate, depending upon the needs of the consumer.

The topical compositions and microbicidal methods using HDP-TFV have also been described in PCT Publication Nos. WO 2009/094191 and WO 2009/094190, which are incorporated by reference in their entireties.

### Formulations

The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

While it is possible for the active ingredients to be administered alone it is preferably to present them as pharmaceutical formulations. The formulations, both for veterinary and for human use, of the present invention comprise at least one active ingredient, as above defined, together with one or more pharmaceutically acceptable carriers (excipients, diluents, etc.) thereof and optionally other therapeutic ingredients. The carrier(s) must be “acceptable” in the sense of being compatible with the other ingredients of the formulation and not deleterious to the recipient thereof.

For infections of the eye or other external tissues e.g. mouth and skin, the formulations are, in some embodiments, applied as a topical ointment or cream containing the active ingredient(s) in an amount of, for example, 0.005 to 20% w/w (including active ingredient(s) in a range between 0.05% and 20% in increments of 0.05% w/w such as 0.6% w/w, 0.65% w/w, 0.7% w/w), in some embodiments, 0.05 to 15% w/w and in other embodiments, 0.05 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. In some embodiments, a hydrophilic emulsifier is included together with a lipophilic emulsifier which acts as a stabilizer. In some embodiments, it includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the present invention include TWEEN®60, SPAN®80, cetostearyl alcohol, benzyl alcohol, myristyl alcohol, glyceryl mono-stearate and sodium lauryl sulfate.

The choice of suitable oils or fats for the formulation is based on achieving the desired cosmetic properties, since the solubility of the active compound in most oils likely to be used in pharmaceutical emulsion formulations is very low. In some embodiments, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. In some embodiments, the active ingredient is present in such formulations in a concentration of 0.1 to 20%. In some embodiments, the active ingredient is present in a concentration of 0.1 to 10%. In some embodiments, the active ingredient is present in a concentration of about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, i.e. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents such as pentamidine for treatment of pneumocystis pneumonia.

Formulations suitable for vaginal administration may be presented as pessaries, rings, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

Compounds described herein may be used to provide controlled release pharmaceutical formulations containing as active ingredient one or more compounds of the invention (“controlled release formulations”) in which the release of the active ingredient can be controlled and regulated to allow less frequent dosing or to improve the pharmacokinetic or toxicity profile of a given invention compound. Controlled release formulations adapted for oral administration in which discrete units comprising one or more compounds of the invention can be prepared according to conventional methods. Controlled release formulations may be employed for the treatment or prophylaxis of various microbial infections particularly human bacterial, human parasitic protozoan or human viral infections caused by microbial species including Plasmodium, Pneumocystis, herpes viruses (CMV, HSV 1, HSV 2, VZV, and the like), retroviruses, adenoviruses and the like. The controlled release formulations can be used to treat HIV infections and related conditions such as tuberculosis, malaria, pneumocystis pneumonia, CMV retinitis, AIDS, AIDS-related complex (ARC) and progressive generalized lymphadeopathy (PGL), and AIDS-related neurological conditions such as multiple sclerosis, and tropical spastic paraparesis. Other human retroviral infections that may be treated with the controlled release formulations according to the invention include Human T-cell Lymphotropic virus and HIV-2 infections. The invention accordingly provides pharmaceutical formulations for use in the treatment or prophylaxis of the above-mentioned human or veterinary conditions and microbial infections.

### Pharmacokinetic Enhancers

The compounds described herein may be employed in combination with pharmacokinetic enhancers (sometimes also referred to as “booster agents”). One aspect of the invention provides the use of an effective amount of an enhancer to enhance or “boost” the pharmacokinetics of a compound of the invention. An effective amount of an enhancer, for example, the amount required to enhance an active compound or additional active compound of the invention, is the amount necessary to improve the pharmacokinetic profile or activity of the compound when compared to its profile when used alone. The compound possesses a better efficacious pharmacokinetic profile than it would without the addition of the enhancer. The amount of pharmacokinetic enhancer used to enhance the potency of the compound is, preferably, subtherapeutic (e.g., dosages below the amount of booster agent conventionally used for therapeutically treating infection in a patient). An enhancing dose for the compounds of the invention is subtherapeutic for treating infection, yet high enough to effect modulation of the metabolism of the compounds of the invention, such that their exposure in a patient is boosted by increased bioavailability, increased blood levels, increased half life, increased time to peak plasma concentration, increased/faster inhibition of HIV integrase, RT or protease and/or reduced systematic clearance. One example of a pharmacokinetic enhancer is RITONAVIR™ (Abbott Laboratories).

### EXAMPLES

### Example I

HDP-TFV K⁺ salt and tenofovir was solubilized at 40 mM and 10 mM in water, respectively, and stored at −20° C. AZT was solubilized at 25 mM in water.

### Co-Culture Assay for HTLV-I

PBMC Preparation: Fresh human peripheral blood mononuclear cells (PBMCs), obtained from Biological Specialty Corporation, Bristol, Pa., were confirmed as seronegative for HIV and HBV. Depending on the volume of the donor blood received, the leukophoresed blood cells were washed several times with PBS. After washing, the leukophoresed blood was diluted 1:1 with Dulbecco's phosphate buffered saline (PBS) and layered over 15 mL of Ficoll-Hypaque density gradient in a 50 mL conical centrifuge tube. These tubes were then centrifuged for 30 min at 600 g. Banded PBMCs were gently aspirated from the resulting interface and subsequently washed three times with PBS by low speed centrifugation. After the final wash, cells were enumerated by trypan blue dye exclusion and re-suspended at 1×10⁶ cells/mL in RPMI 1640 with 15% Fetal Bovine Serum (FBS), 2 mmol/L L-glutamine, 2 μg/mL phytohemagglutinin (PHA-P), 100 Units/mL penicillin and 100 μg/mL streptomycin and allowed to incubate for 48-72 hr at 37° C.

After incubation, PBMCs were centrifuged and resuspended in PBMC medium (RPMI 1640 with 15% FBS, 2 mmol/L L-glutamine, 100 U/mL penicillin, 100 μg/mL streptomycin and 20 U/mL recombinant human IL-2). The cultures were then maintained for the remainder of the experiment by exchange of half the culture volume with fresh IL-2 containing tissue culture medium every 3 days. Assays were initiated with PBMCs that had been induced to proliferate for 72 hr. Stimulated PBMCs from two donors were pooled together to minimize the variability between individual donors and 8×10⁶ cells were resuspended in 9 mL of fresh tissue culture medium per T25 flask.

HDP-TFV was evaluated at concentrations of 0.04, 0.2, 1 and 10 μM. AZT and tenofovir were evaluated at concentrations of 0.1, 1, 5 and 25 μM. HDP-TFV, AZT, or tenofovir were added to the PBMCs 10 hours prior to infection.

MT-2 Preparation: MT-2 cells were obtained from the NIH AIDS Research and Reference Reagent Program and passaged in T-75 flasks in RPMI 1640 medium, supplemented with 10% heat inactivated fetal bovine serum, 2 mmol/L L-glutamine, 100 U/ml penicillin and 100 μg/ml streptomycin. Total cell and viability quantification were performed using hemocytometer and trypan blue dye exclusion. The cells were incubated in 10 mL of 200 μg/mL mitomycin C for 1 hour at 37° C./5% CO₂, washed 3 times in Dulbecco's phosphate buffered saline (DPBS), and resuspended in PBMC medium at 1.6×10⁶ cells per mL. One mL of treated MT-2 cells was added to each T25 flask, except for the PBMC only control.

Co-Culture for HTLV-I Replication: Cell cultures were incubated at 37° C./5% CO₂ for four weeks. Cell viability and density was monitored by trypan blue dye exclusion test. Cell density was readjusted to 8×10⁵ cells per mL on days 3, 7, 10, 14, 21 and 28. Compound was added with the half-volume fresh medium exchange on days 3, 7, and 10. Supernatant was collected on days 14, 21 and 28 for measurement of HTLV-I virus replication by p19 Gag ELISA. Cell samples were also collected on days 14 and 28 for genomic DNA extraction and PCR analysis of HTLV-I proviral DNA. Untreated PBMCs co-cultured with MT-2 cells, PBMCs alone, and mitomycin C-treated MT-2 cells alone were cultured in parallel as controls.

HTLV-I p19 Gag ELISA: ELISA was performed to quantify p19 in cell-free supernatants according to the manufacturer's instructions (ZeptoMetrix, Buffalo, N.Y.). Briefly, 50 μL of the kit p19 standard was added to 950 μL assay diluent in microtiter tubes and serially diluted 1:2. After washing the microtiter plate with 1× plate wash buffer, 200 μL of the diluted standards were added in duplicate to the coated wells. In duplicate, 200 μL of media was added as negative controls. Cell culture supernatants were diluted 1:9 with media and mixed with 50 μL of lysis buffer in duplicate. Two hundred microliters of diluted sample was added to the coated wells and incubated at 37° C. for 2 hours. Following the incubation, the plate was washed six times with 300 μL of wash buffer provided with the kit. One hundred twenty microliters of HTLV-I detector antibody was added to 12 mL of assay diluent, mixed and 100 μL was added to each well, except A1 and A2. The plate was incubated at 37° C. for 1 hour then washed as above. One hundred twenty microliters of peroxidase was added to 12 mL of assay diluent, mixed and 100 μL microliters was added to each well, except A1 and A2. The plate was incubated at 37° C. for 1 hour then washed. One hundred twenty microliters of substrate was added to 12 mL of substrate diluent and mixed. Substrate solution (100 μL) was added to the entire plate. The plate was incubated at room temperature for 30 minutes protected from light then 100 μL of stop solution was added to each well. Plates were read spectrophotometrically at wavelengths 450 nm within 30 minutes of adding stop solution. The quantity of free HTLV-I p19 antigen in the sample was determined by comparing its absorbance to that of the control standards.

PCR of Proviral HTLV-I DNA: The presence of HTLV-I proviral DNA at 2 and 4 weeks in treated and untreated PBMC cells was examined using a PCR assay. Total DNA was extracted from frozen cell pellets using Qiagen DNEasy Blood and Tissue kits according to the manufacturer's recommended method for extraction from cultured cells. DNA was eluted in 200 μl of Buffer AE and the concentration was determined by absorbance at 260 nm in a Spectramax 386 Plus plate reader. Fifty nanograms of extracted DNA was subjected to two separate PCR amplifications using primer sets for HTLV-I and human GAPDH. Amplifications were performed using PCR primer sets designed at ImQuest BioSciences and synthesized by IDT (Coraville, Id.). Amplification of HTLV-I proviral DNA was performed using 50 ng of extracted DNA in a 25 μl reaction volume with TaqPro complete (Denville Scientific, Metuchen, N.J.) and DNA oligonucleotide primers (0.2 μM each) HTLV-3281-F (5′-AAC TTC AAG CCC TAC TTG GCG AGA-3′[SED ID NO.: 1]) and HTLV-3666-R (5′-TGT ATG GTT TGG CAG AGT AGC CCA-3′ [SED ID NO.: 2]). Amplification of human GAPDH DNA sequences was performed using 50 ng of extracted DNA in a 25 μl reaction volume with TaqPro complete and DNA oligonucleotide primers (0.2 μM each) hGAPDH-gF1 (5′-GAA GGA AAT GAA TGG GCA GCC GTT-3′ [SED ID NO.: 3]) and GAPDH-gR1 (5′-ATT TGC CAA GTT GCC TGT CCT TCC-3′ [SED ID NO.: 4]). Amplification conditions for both HTLV-I and GAPDH consisted of an initial denaturation step of 95° C. for 5 min followed by 40 cycles of 95° C. for 30 sec, 62° C. for 30 sec, 72° C. for 45 sec and a final extension of 72° C. for 5 min. The amplified DNA products were evaluated by agarose gel electrophoresis and ethidium bromide staining.

### Results

Anti-HTLV-I Evaluations: HDP-TFV K⁺ salt and tenofovir were evaluated for HTLV-I inhibition in human PBMCs co-cultured with mitomycin C treated MT-2 cells at four concentrations of each compound. AZT was evaluated in parallel as a control compound. The results of the anti-HTLV-I p19 ELISA are summarized in Table 2. The graphical representation of these data compares the antiviral efficacy expressed as a percent of the control (untreated PBMCs cultured with MT-2 cells). Cell viability was monitored by trypan blue dye exclusion. AZT and tenofovir were not toxic to the infected PBMCs at concentrations up to 25 μM at 2, 3, or 4 weeks post infection. HDP-TFV was increasingly toxic to the infected PBMCs with TC₅₀ values of 6.3 and 1.0 μM at 3 and 4 weeks post infection, respectively.

AZT and tenofovir yielded similar results with no antiviral activity at 2 weeks post infection up to 25 μM. AZT yielded EC₅₀ values of 3.2 and 2.6 μM at 3 and 4 weeks post infection, respectively. Tenofovir yielded EC₅₀ values of 10.3 and 4.5 μM at 3 and 4 weeks post infection, respectively. HTLV-I infected PBMCs yielded EC₅₀ values of 6.8, 0.9 and 0.2 μM when treated with HDP-TFV for 2, 3 and 4 weeks, respectively.

TABLE 2
HTLV-I Antiviral Evaluation by p19 ELISA

| | HTLV-I EC₅₀ (μM) | | | HTLV-I TC₅₀ (μM) | | |
|---|---|---|---|---|---|---|
| Compound | 2 wks | 3 wks | 4 wks | 2 wks | 3 wks | 4 wks |
| AZT | >25.0 | 3.2 | 2.6 | >25.0 | >25.0 | >25.0 |
| Tenofovir | >25.0 | 10.3 | 4.5 | >25.0 | >25.0 | >25.0 |
| HDP-TFV | 6.8 | 0.9 | 0.2 | >10.0 | 6.3 | 1.0 |

HTLV-I Antiviral Evaluation by Proviral DNA: HTLV-I and human GAPDH sequences were amplified using control DNA specimen from uninfected PBMCs and HTLV-I infected PBMCs from co-culture with MT-2 cells.

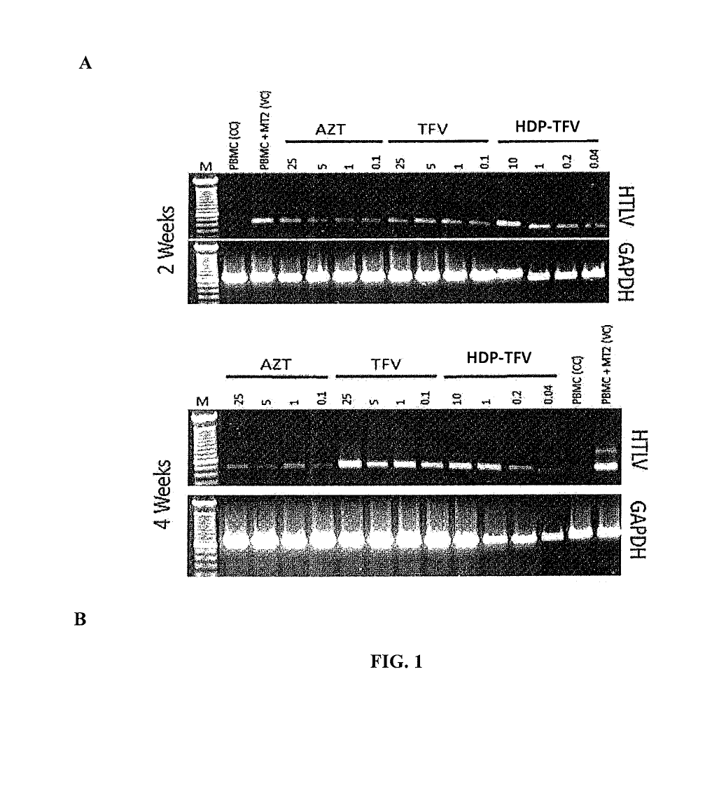

The intensity of the GAPDH product depended on which compound was used, i.e., whether AZT, tenofovir, or HDP-TFV was used, and the duration of their use. HTLV-I primers amplified DNA from the HTLV-I infected cultures but not from uninfected PBMC cultures, demonstrating specificity of the primers for HTLV-I amplification. See FIG. 1. At two and four weeks, at all concentrations of AZT or tenofovir, the relative intensity of amplified HTLV-I was either equal or less than the intensity of amplified HTLV-I from infected controls (PBMC+MT-2 cells). See id. (compare lanes marked as AZT & TFV, with lane marked as PBMC+MT-2). Amplification of GAPDH was similar in each of these samples. Inhibition of HTLV proviral DNA accumulation was apparent following four weeks of treatment with the lower concentrations of HDP-TFV.

AZT, tenofovir, and HDP-TFV, were evaluated for anti-HTLV-I inhibition in a co-culture assay using human PBMCs infected with mitomycin C treated MT-2 cells. Infected cells were cultured for four weeks with quantification of p19 Gag antigen in the supernatant at weeks 2, 3 and 4 by ELISA and quantification of integrated proviral DNA measured by PCR at weeks 2 and 4. AZT and tenofovir inhibited HTLV-I replication following 2 weeks in culture as measured by p19 ELISA at concentrations above 10 M. Quantitative PCR indicates less integration of proviral DNA when infected cells are cultured in the presence of AZT at concentrations above 0.1 μM or 25 μM tenofovir. HDP-TFV inhibited HTLV-I virus replication from infected PBMCs as early as 2 weeks post-infection at concentrations above 7 μM with greater inhibition at 3 and 4 weeks of culture; however, the compound was increasingly toxic as fresh HDP-TFV was added to the cultures up to day 10 and compound accumulated in the cells.

### Example II

### Compounds

HDP-CDV and cidofovir were solubilized at 10 mM in water and 40 mM in DMSO, respectively. HDP-CDV can be prepared according to procedures known in the art. See e.g., US Patent Publication No. 2007/0003516; the contents of which are incorporated by reference herein. Solubilized HDP-CDV was stored at room temperature. AZT was solubilized at 25 mM in water. Solubilized AZT and cidofovir were stored at −20° C.

### Co-Culture Assay for HTLV-I

PBMC Preparation: Fresh human peripheral blood mononuclear cells (PBMCs) were obtained from Biological Specialty Corporation, Bristol, Pa., and were determined to be seronegative for HIV and HBV. Depending on the volume of the donor blood received, the leukophoresed blood cells were washed several times with PBS. After washing, the leukophoresed blood was diluted 1:1 with Dulbecco's phosphate buffered saline (PBS) and layered over 15 mL of Ficoll-Hypaque density gradient in a 50 mL conical centrifuge tube. These tubes were then centrifuged for 30 min at 600 g. Banded PBMCs were gently aspirated from the resulting interface and subsequently washed three times with PBS by low speed centrifugation. After the final wash, cells were enumerated by trypan blue dye exclusion and re-suspended at 1×10⁶ cells/mL in RPMI 1640 with 15% Fetal Bovine Serum (FBS), 2 mmol/L L-glutamine, 2 μg/mL phytohemagglutinin (PHA-P), 100 Units/mL penicillin and 100 μg/mL streptomycin and allowed to incubate for 48-72 hr at 37° C.

After incubation, PBMCs were centrifuged and resuspended in PBMC medium (RPMI 1640 with 15% FBS, 2 mmol/L L-glutamine, 100 U/mL penicillin, 100 μg/mL streptomycin and 20 U/mL recombinant human IL-2). The cultures were then maintained for the remainder of the experiment by exchange of half the culture volume with fresh IL-2 containing tissue culture medium every 3 days. Assays were initiated with PBMCs that had been induced to proliferate for 72 hr. Stimulated PBMCs from two donors were pooled together to minimize the variability between individual donors and 8×10⁶ cells were resuspended in 9 mL of fresh tissue culture medium per T25 flask.

HDP-CDV was evaluated at concentrations of 0.04, 0.2, 1 and 10 μM. Cidofovir was evaluated at concentrations of 1, 5, 25 and 100 μM. AZT was evaluated at concentrations of 0.1, 1, 5 and 25 μM. Compounds were added to the PBMCs 10 hours prior to infection.

MT-2 Preparation: MT-2 cells were obtained from the NIH AIDS Research and Reference Reagent Program and passaged in T-75 flasks in RPMI1640 medium supplemented with 10% heat inactivated fetal bovine serum, 2 mmol/L L-glutamine, 100 U/mI penicillin and 100 μg/ml streptomycin. Total cell and viability quantification were performed using a hemocytometer and trypan blue dye exclusion. The cells were incubated in 10 mL of 200 μg/mL mitomycin C for 1 hour at 37° C./5% CO₂, washed 3 times in Dulbecco's phosphate buffered saline (DPBS), and resuspended in PBMC medium at 1.6×10⁶ cells per mL. One mL of treated MT-2 cells was added to each T25 flask, except for the PBMC only control.

Co-Culture for HTLV-I Replication: Cell cultures were incubated at 37° C./5% CO2 for four weeks. Cell viability and density was monitored by trypan blue dye exclusion test. Cell density was readjusted to 8×10⁵ cells per mL on days 3, 7, 10, 14, 21 and 28. Compound was added with the half-volume fresh medium exchange on days 3, 7, and 10. Supernatant was collected on days 14, 21 and 28 for measurement of HTLV-I virus replication by p19 Gag ELISA. Cell samples were also collected on days 14 and 28 for genomic DNA extraction and PCR analysis of HTLV-I proviral DNA. Untreated PBMCs co-cultured with MT-2 cells, PBMCs alone, and mitomycin C-treated MT-2 cells alone were cultured in parallel as controls.

HTLV-I p19 Gag ELISA: ELISA to quantify p19 in cell-free supernatants was performed according to the manufacturer's instructions (ZeptoMetrix, Buffalo, N.Y.). Briefly, 50 μL of the kit p19 standard was added to 950 μL assay diluent in microtiter tubes and serially diluted 1:2. After washing the microtiter plate with 1× plate wash buffer, 200 μL of the diluted standards were added in duplicate to the coated wells. In duplicate, 200 μL of media was added as negative controls. Cell culture supernatants were diluted 1:9 with media and mixed with 50 μL of lysis buffer in duplicate. Two hundred microliters of diluted sample was added to the coated wells and incubated at 37° C. for 2 hours.

Following the incubation, the plate was washed six times with 300 μL of wash buffer provided with the kit. One hundred twenty microliters of HTLV-I detector antibody was added to 12 mL of assay diluent, mixed and 100 μL was added to each well, except A1 and A2. The plate was incubated at 37° C. for 1 hour then washed as above. One hundred twenty microliters of peroxidase was added to 12 mL of assay diluent, mixed and 100 μL microliters was added to each well, except A1 and A2. The plate was incubated at 37° C. for 1 hour then washed. One hundred twenty microliters of substrate was added to 12 mL of substrate diluent and mixed. Substrate solution (100 μL) was added to the entire plate. The plate was incubated at room temperature for 30 minutes protected from light then 100 μL of stop solution was added to each well.

Plates were read spectrophotometrically at wavelengths 450 nm within 30 minutes of adding stop solution. The quantity of free HTLV-I p19 antigen in the sample was determined by comparing its absorbance to that of the control standards.

PCR of Proviral HTLV-I DNA: The presence of HTLV-I proviral DNA at 2 and 4 weeks in treated and untreated PBMC cells was examined using a PCR assay. Total DNA was extracted from frozen cell pellets using Qiagen DNEasy Blood and Tissue kits according to the manufacturer's recommended method for extraction from cultured cells. DNA was eluted in 200 μl of Buffer AE and the concentration was determined by absorbance at 260 nm in a Spectramax 386 Plus plate reader. Fifty nanograms of extracted DNA was subjected to two separate PCR amplifications using primer sets for HTLV-I and human GAPDH. Amplifications were performed using PCR primer sets designed at ImQuest BioSciences and synthesized by IDT (Coraville, Id.). Amplification of HTLV-I proviral DNA was performed using 50 ng of extracted DNA in a 25 μl reaction volume with TaqPro complete (Denville Scientific, Metuchen, N.J.) and DNA oligonucleotide primers (0.2 μM each) HTLV-3281-F (5′-AAC TTC AAG CCC TAC TTG GCG AGA-3′ [SED ID NO.: 5]) and HTLV-3666-R (5′-TGT ATG GTT TGG CAG AGT AGC CCA-3′ [SED ID NO.: 6]). Amplification of human GAPDH DNA sequences was performed using 50 ng of extracted DNA in a 25 μl reaction volume with TaqPro complete and DNA oligonucleotide primers (0.2 μM each) hGAPDH-gF1 (5′-GAA GGA AAT GAA TGG GCA GCC GTT-3′ [SED ID NO.: 7]) and GAPDH-gR1 (5′-ATT TGC CAA GTT GCC TGT CCT TCC-3′ [SED ID NO.: 8]). Amplification conditions for both HTLV-I and GAPDH consisted of an initial denaturation step of 95° C. for 5 min followed by 40 cycles of 95° C. for 30 sec, 62° C. for 30 sec, 72° C. for 45 sec and a final extension of 72° C. for 5 min. The amplified DNA products were evaluated by agarose gel electrophoresis and ethidium bromide staining.

### Results

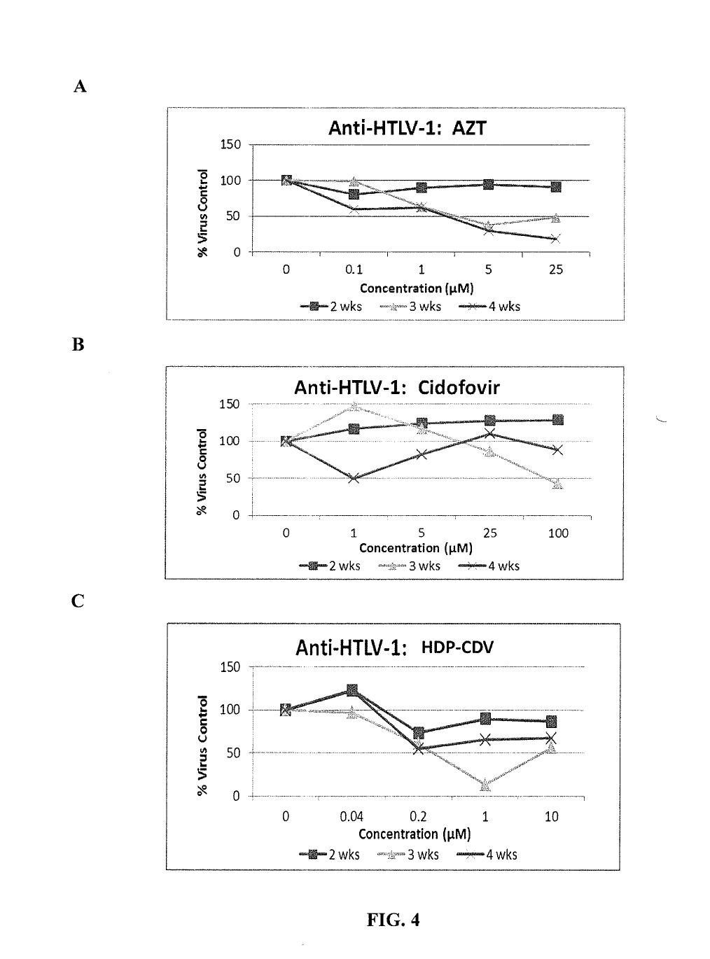

Anti-HTLV-I Evaluations: HDP-CDV and cidofovir were evaluated for HTLV-I inhibition in human PBMCs co-cultured with mitomycin C treated MT-2 cells at four concentrations of each compound. AZT was evaluated in parallel as a control compound. The results of the anti-HTLV-I p19 ELISA are summarized in Table 3. The graphical representation of these data shown in FIG. 4 compares the antiviral efficacy expressed as a percent of the control (untreated PBMCs cultured with MT-2 cells). Cell viability was monitored by trypan blue dye exclusion. AZT was not toxic to the infected PBMCs at concentrations up to 25 μM at 2, 3, or 4 weeks post infection. Cidofovir was slightly toxic at 100 μM following 4 weeks in culture. HDP-CDV was increasingly toxic to the infected PBMCs with TC₅₀ values of 7.5, 3.2 and 1.4 μM at 2, 3 and 4 weeks post infection, respectively.

AZT yielded EC₅₀ values of 3.2 and 2.6 μM at 3 and 4 weeks post infection, respectively. Cidofovir and HDP-CDV yielded EC₅₀ values of 87.8 and 0.4 μM at 3 weeks post infection, respectively, but did not suppress virus replication greater than 50% following 2 and 4 weeks co-culture. Moderate antiviral activity was demonstrated from HDP-CDV concentrations of 0.2 μM and above following four weeks of infected PBMC culture; however, inhibition may be attributed to observed toxicity.

TABLE 3
HTLV-I Antiviral Evaluation by p19 ELISA

| | HTLV-I EC₅₀ (μM) | | | HTLV-I TC₅₀ (μM) | | |
|---|---|---|---|---|---|---|
| Compound | 2 wks | 3 wks | 4 wks | 2 wks | 3 wks | 4 wks |
| AZT | >25.0 | 3.2 | 2.6 | >25.0 | >25.0 | >25.0 |
| Cidofovir | >100.0 | 87.8 | >100.0 | >100.0 | >100.0 | 80.0 |
| HDP-CDV | >10.0 | 0.4 | >10.0 | 7.5 | 3.2 | 1.4 |

HTLV-I Antiviral Evaluation by Proviral DNA: The amplified DNA products were evaluated by agarose gel electrophoresis and ethidium bromide staining. HTLV-I primers only amplified DNA from the HTLV-I infected cultures and not from PBMC cultures, demonstrating specificity of the primers for HTLV-I amplification. The relative intensity of HTLV amplification appeared to be equal or less to infected controls with all concentrations of AZT at two and four weeks. Amplification of GAPDH was similar in each of these samples. Compound HDP-CDV inhibited the accumulation of HTLV proviral DNA after two weeks of treatment at a concentration equal to or greater than 0.2 μM and at all concentrations following four weeks of treatment. Inhibition of HTLV-I proviral DNA accumulation was also apparent following two weeks of treatment with 100 μM cidofovir (CDV) and following four weeks of treatment with 25 and 100 μM when compared to the untreated control.

AZT, cidofovir, HDP-CDV, were evaluated for anti-HTLV-I inhibition in a co-culture assay using human PBMCs infected with mitomycin C treated MT-2 cells. Infected cells were cultured for four weeks with quantification of p19 Gag antigen in the supernatant at weeks 2, 3 and 4 by ELISA and quantification of integrated proviral DNA measured by PCR at weeks 2 and 4. AZT inhibited HTLV-I replication following 2 weeks in culture as measured by p19 ELISA at concentrations above 3 μM. Quantitative PCR indicates less integration of proviral DNA when infected cells are cultured in the presence of AZT at concentrations above 0.1 μM.

Cidofovir and HDP-CDV inhibited HTLV-I virus replication from infected PBMCs at 3 weeks post-infection at concentrations at 100 μM and above 3 μM, respectively. Greater than 50% inhibition of virus replication measured by p19 ELISA was not reached for cidofovir or HDP-CDV at 2 or 4 weeks post-infection. HDP-CDV was increasingly toxic as fresh compound was added to the cultures up to day 10, which may account for the decreased integration of proviral DNA at 2 and 4 weeks post-infection, though GAPDH levels were similar to the PBMC controls at 2 weeks of culture and slightly lower at 4 weeks.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed and that modifications to the disclosed embodiments, as well as other embodiments, are intended to be included within the scope of the appended claims. The invention is defined by the following claims, with equivalents of the claims to be included therein.

What is claimed is:

1. A pharmaceutical composition for treating a viral infection or viral disease, comprising a compound having a formula: 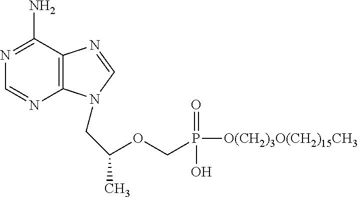
or a pharmaceutically acceptable salt thereof, wherein the viral infection or viral disease is treated in about three weeks after administration.

2. The composition of claim 1, wherein the compound decreases viral replication.

3. The composition of claim 1, wherein the viral infection is human T cell leukemia virus-1 (HTLV-I) infection.

4. A method for treating a viral infection or viral disease in a subject, the method comprising administering to the subject a composition of claim 1, wherein the compound is effective in treating the viral infection or viral disease in about three weeks after administration.

5. The method of claim 4, wherein the method results in decreasing viral replication.

6. The method of claim 4, wherein the virus is a retrovirus.

7. The method of claim 4, wherein said viral infection or viral disease is an infection or disease of a human retrovirus selected from the group consisting of HIV-1, HIV-2, HTLV-I and HTLV-II.

8. The method of claim 4, wherein said subject is a human being.

9. The method of claim 8, wherein the administration is before acute viral infection.

10. The method of claim 8, wherein said composition is administered before seroconversion.

11. The method of claim 8, wherein said composition is administered after serocoversion.

12. A method for inhibiting replication of reverse transcriptase dependent virus in animal cells, comprising administering to said cells a composition comprising the compound of having a formula: 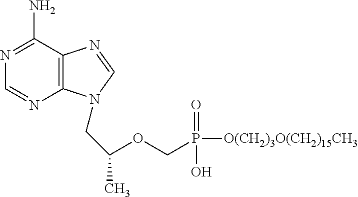
or pharmaceutically acceptable salt thereof.

13. The method of claim 12, wherein the compound is administered to cells in vivo.

14. The method of claim 12, wherein said animal cells are mammalian cells.

15. The method of claim 12, wherein the virus is a retrovirus.

16. The method of claim 12, wherein said virus is a human retrovirus selected from the group consisting of HIV-1, HIV-2, HTLV-I and HTLV-II and said cells are human cells.

17. The method of claim 12, wherein said composition is administered to a human being before acute viral infection.

18. The method of claim 12, wherein said composition is administered to a human being before seroconversion.

19. The method of claim 12, wherein said composition is administered to a human being after serocoversion.
